(19) United States
(12) Patent Application Publication
(10) Pub. No.: US 2023/0078811 A1
(43) Pub. Date: Mar. 16, 2023

(54) USE OF DISTILLABLE VOLATILE SALT FOR THE PRETREATMENT OF BIOMASS

(71) Applicants: The Regents of the University of California, Oakland, CA (US); National Technology & Engineering Solutions of Sandia, LLC, Albuquerque, NM (US)

(72) Inventors: Ezinne C. ACHINIVU, Alexandria, VA (US); John M. GLADDEN, Alameda, CA (US); Hemant CHOUDHARY, Emeryville, CA (US); Blake A. SIMMONS, San Francisco, CA (US)

(21) Appl. No.: 17/800,871

(22) PCT Filed: Feb. 18, 2021
(86) PCT No.: PCT/US21/18630

§ 371 Date: Aug. 18, 2022

Related U.S. Application Data
(60) Provisional application No. 62/978,771, filed on Feb. 19, 2020.

(51) Int. Cl. C12P 19/02; C12P 19/14

(52) U.S. Cl. CPC C12P 19/02; C12P 19/14

(57) ABSTRACT

The present invention provides for a method to produce a sugar from a biomass, the method comprising: (a) providing a first mixture comprising a solubilized biomass and a distillable acid-base conjugate salt (DABCS) or deep eutectic solvent (DES), wherein (i) the DABCS is a protic ionic liquid (PIL) or a protic salt comprising a DABCS cation and a DABCS anion, and (ii) the DES is any combination of Lewis or Brønsted acid and base comprising any anionic and/or cationic species that have sufficient vapor pressure so that it can be readily distilled; and (b) distilling at least part of the DABCS from the first mixture in order to separate the at least part of the DABCS from the first mixture.

### CROSS REFERENCE TO RELATED APPLICATIONS

This application claims priority to U.S. Provisional Patent Application Ser. No. 62/978,771, filed Feb. 19, 2020, which is incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of using distillable volatile salt for biomass pretreatment.

### BACKGROUND OF THE INVENTION

Biofuels and bioproducts derived from sustainable feedstocks are considered a potential solution to address the challenges associated with human population growth. For efficient biofuel production, the biochemical conversion of lignocellulosic biomass has been frequently discussed in terms of process optimization as well as the reaction mechanism of various thermochemical processing (e.g., pretreatment) and biochemical conversion (e.g., enzymatic hydrolysis and fermentation). Current challenges to the realization of an affordable and scalable biomass conversion technology are those associated with complicated process designs, difficulties associated with efficient solvent recycle, and water consumption.

### SUMMARY OF THE INVENTION

The present invention provides for using a distillable volatile salt, or a mixture thereof, for the pretreatment of biomass. In some embodiments, the distillable volatile salt is an ionic liquid or a deep eutectic solvent, or a mixture thereof.

The present invention provides for a method to produce a sugar from a biomass, the method comprising: (a) providing a first mixture comprising a solubilized biomass and a distillable acid-base conjugate salt (DABCS) or deep eutectic solvent (DES), wherein (i) the DABCS is a protic ionic liquid (PIL) or a protic salt comprising a DABCS cation and a DABCS anion, and (ii) the DES is any combination of Lewis or Brønsted acid and base comprising any anionic and/or cationic species that have sufficient vapor pressure so that it can be readily distilled; (b) distilling at least part of the DABCS from the first mixture in order to separate the at least part of the DABCS from the first mixture; (c) optionally introducing an enzyme and/or a microbe to the first mixture such that the enzyme and/or microbe produce a sugar from the solubilized biomass; and, (d) optionally the sugar is separated from the first mixture.

In some embodiments, the method further comprises (e) introducing at least part of the DABCS separated in the (b) distilling step to the first mixture in step (a).

In some embodiments, the method further comprises (f) introducing more biomass to the first mixture in step (a).

The present invention provides for a first mixture comprising a biomass and a distillable acid-base conjugate salt (DABCS) comprising a DABCS cation, and a DABCS anion.

The present invention provides for a system comprising a reactor vessel, a condenser, a collection chamber, one or more cold traps, and low pressure source; wherein the reactor vessel is in fluid communication with a condenser, wherein the condenser is in fluid communication with (i) the collection chamber and (ii) the low pressure source via the one or more cold traps. In some embodiments, the reactor vessel comprises a double wall and/or a mixer or propeller. In some embodiments, the condenser has a temperature of about 0° C., −5° C., −10° C., −15° C., −20° C., −25° C., −30° C., −35° C., −40° C., −45° C., or −50° C. In some embodiments, each cold trap has a temperature lower than the temperature of the condenser, such as about −60° C., −65° C., −70° C., −75° C., −80° C., −85° C., or −90° C. In some embodiments, the low pressure source is a vacuum pump, for example the vacuum pump is creating a pressure of about 1 m torr.

In some embodiments, the reaction vessel contains the first mixture of the present invention. In some embodiments, the first mixture has a solid loading of about 5%, 10%, 15%, or 20%. In some embodiments, the first mixture is heated to a temperature of about 100° C., 110° C., 120° C., 130° C., 140° C., 150° C., or 160° C., or any temperature between any two of the preceding temperatures. In some embodiments, the first mixture is incubated for a first time for about 1 h, 2 h, 3 h, 4 h, or 5 h.

In some embodiments, after the first incubation, the first mixture is subjected to a distilling step, wherein the first mixture is heated to a temperature of about 100° C., 110° C., 120° C., 130° C., 140° C., 150° C., or 160° C., or any temperature between any two of the preceding temperatures, and a low pressure (produced by the low pressure source) applied to the reaction vessel. Distillable IL/DES/solvent (from the liquid phase in the first mixture) is converted into the gaseous phase and is conveyed to the condenser, where it converts back into the liquid phase and is collected in the collection chamber.

In some embodiments, after the distilling step, the temperature is lowered to about 25° C., 30° C., 35° C., 37° C., or 40° C., a suitable enzyme or microbe, or mixture thereof, is added to the first mixture, and incubated for a second time for about 24 h, 48 h, 72 h, 96 h, or 120 h. The second incubation results in the production of one or more sugar monomers, such as hexoses and/or pentoses.

The present invention provides for compositions and methods described herein.

In some embodiments, the compositions and methods further comprise steps, features, and/or elements described in U.S. patent application Ser. No. 16/737,724, hereby incorporated by reference in its entirety.

In some embodiments, the method further comprises one or more of the following: (a) introducing a biomass and a deep eutectic solvent (DES), or mixture thereof, into a vessel to form a one-pot composition, wherein the DES, or mixture thereof, solubilizes the biomass; (b) introducing an enzyme and/or a microbe to the one-pot composition such that the enzyme and/or microbe produce a biofuel and/or chemical compound from the solubilized biomass; and, (c) optionally separating the biofuel and/or chemical compound from the one-pot composition. In some embodiments, the introducing steps (a) and (b), and optionally the separating step (c), are continuous.

In some embodiments, the method, or one-pot method, does not require any solid-liquid separation step. In some embodiments, the one-pot method does not require adjustment of the pH level in the one-pot composition. In some embodiments, the one-pot method does not require any dilution, or addition of water or medium, after pretreatment and/or before saccharification and fermentation. In some embodiments, the reaction of the enzyme and the growth of the microbe occur in the same one-pot composition. In some embodiments, the DES, or mixture thereof, is renewable as it can be continuous in use. In some embodiments, the one-pot method can produce a yield of sugar that is equal to or more than about 50%, 55%, 60%, 65%, 70%, 75%, or 80%, or any other value described herein.

In some embodiments, the one-pot biomass pretreatment, saccharification, and fermentation with bio-compatible deep eutectic solvents (DESs). The used bio-compatible DESs are tested for microbial, such as yeast, compatibility and toxicity. The pretreatment efficacy of the selected DESs are tested. The uses of the DESs for biomass processing eliminates the need to remove any solvent after biomass pretreatment, thus making the one-pot approach possible.

In some embodiments, using bio-compatible DESs enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

This invention provides for the use of volatile protic salts (such as ionic liquids) and deep eutectic solvents to develop an integrated biomass pretreatment approach that combines effective IL-based pretreatment with a simplistic and energy efficient IL recovery/recycling method. This novel method enables the cost-effective production of IL-based fermentable sugars—a major hurdle for producing commercially viable bioenergy from waste biomass. By integrating a vacuum distillation for the salt recovery/recycling into the overall pretreatment process, the development of the most favorable process configuration is facilitated that maximizes both IL recovery, along with, pretreatment effectiveness (such as sugar yield and/or lignin yield). Subsequently different salts can be screened to identify key structural features that enhance the overall process economics.

The utilization of protic ionic salts, such as ionic liquids (ILs) and deep eutectic solvents (DESs), as biomass pretreatment solvents is one of the most effective methods for producing high yields of fermentable sugars for bioenergy production. However, the cost of IL utilization is a significant problem that must be addressed before an affordable IL/DES-based process is commercially viable. Therefore, this invention describes a combined method for performing biomass pretreatment that involves utilizing protic salts that are relatively cheap, effective at deconstructing biomass and most importantly—readily recyclable. In some embodiments, the method comprises an integrated/one-pot approach for carrying out pretreatment, IL recovery/recycle, enzymatic hydrolyses (such as for sugar release) and fermentation sequentially to minimize the need for tedious downstream separation and to maximize biofuel/bioproduct yield.

For this process, a distillable acid-base conjugate salt can be utilized, such as a protic ionic liquid (PM), as well as DESs that can be effortlessly recycled with little energy input to enable their easy recovery. Effective PILs for this process should be recovered at least equal to more than about 85% yield by distilling over vacuum at T≤about 160° C., while releasing at least about 85% fermentable sugars. These PILs (such as hydroxyethylammonium acetate—[Eth][OAc]) are demonstrated to be effective for biomass pretreatment and are also relatively cheap due to their ease of synthesis. To develop this process, it was necessary to develop the most favorable process configuration to maximize both IL recovery, along with, pretreatment effectiveness (such as sugar yield and/or lignin yield). Subsequently several ILs can be screened to identify analogous ILs with similar cations/anions in order to improve the overall process economics. Suitable salts for this process include combinations of organic ammonium-based cations (such as ammonium, hydroxyalkylammonium, dimethylalkylammonium) with organic carboxylic acid-based anions (such as acetic acid derivatives (C1-C8), lactic acid, glycolic acid, as well as, DESs such as ammonium acetate/lactic acid.

Preliminary results show that the PIL—[Eth][OAc] can be recovered at a rate of [˜85-98]% in a one-pot setup depending on the solids loading of the biomass. Additional work needs to be carried out to optimize the process configuration for high recovery yield, as well as, to discover other PILs and/or DESs that may have improved pretreatment efficiency and/or recovery rates.

The invention outlined here provides numerous advantages including one or more of the following: (1) Developing cheaper solvents for biomass pretreatment, (2) Effective at pretreatment (via lignin extraction and reducing biomass recalcitrance), (3) facile recycling and recover of ILs and DESs via vacuum distillation, and (4) Integrated approach for the conversion of biomass to biobased fuels.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. An embodiment for the recovery of distillable IL/solvent.

FIG. 2. A Piping and instrumentation Diagram (P&ID) of an embodiment of a method for the recovery of distillable IL/solvent used in a biomass pretreatment system.

FIG. 3. A P&ID of an embodiment of a method for the recovery of distillable IL/solvent used in a biomass pretreatment system.

FIG. 4. Concentration of pretreating solvent in the solids after pretreatment.

FIG. 5. Analysis of sugars and acids in solids after distillation. “HMF” refers to hydroxymethylfurfural.

FIG. 6. Analysis of aromatics in solids after distillation. “4HBA” refers to 4-hydroxybenzoic acid.

FIG. 7. Sugar release after pretreatment, distillation and saccharification.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. Any Lewis or Brønsted acid and base combination can be used in the invention as long as the combination is distillable.

Typically, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The novel DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DABCS or DES is distillable if the DABCS or DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 15020 C., or 160 ° C., or any temperature between any two of the preceding temperatures. In some embodiments, the method results in the releasing of at least equal to or more than about 60%, 65%, 70%, 75%, 80%, or 85% fermentable sugars.

Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethyl ammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid.

Suitable distillable ionic liquids are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable distillable PIL are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19:3152-3163 (2017), all of which are hereby incorporated by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing steps (a) and (b) together produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps (a) to (c) are continuous, the one-pot composition is in a steady state.

In some embodiments, the DES can be one taught in WO 2018/204424 (Seema Singh et al.), which is hereby incorporated in its entirety by reference.

In some embodiments, all or some of the one-pot composition is further pretreated as follows: the method further comprising: (d) optionally separating the sugar and the lignin in the one-pot composition, (e) depolymerizing and/or converting the lignin into one or more lignin derived monomeric phenol, or a mixture thereof, (f) providing the one or more lignin derived monomeric phenol, or a mixture thereof, in a solution, (g) introducing one or more quaternary ammonium salts, or a mixture thereof, to the solution, (h) heating the solution, such that steps (g) and (h) together result in the synthesis of a DES, (i) optionally forming a DES system from the DES synthesized in step (h), and (j) optionally repeating steps (d) to (i) using the DES system formed in step (i) in the introducing step (a).

In some embodiments, the heating step (h) comprises increasing the temperature of the solution to a value within a range of about 75° C. to about 125° C. In some embodiments, the heating step (h) comprises increasing the temperature of the solution to a value within a range of about 80° C. to about 120° C. In some embodiments, the heating step (h) comprises increasing the temperature of the solution to a value within a range of about 90° C. to about 110° C. In some embodiments, the heating step (h) comprises increasing the temperature of the solution to about 100° C.

In some embodiments, the enzyme is a cellulase. In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Deep eutectic solvents (DESs) share the promising solvent properties of ionic liquids. They show low volatility, wide liquid range, water-compatibility, non-flammability, non-toxicity, biocompatibility and biodegradability. Furthermore, DES can be easily prepared from readily available materials at high purities and low cost compared to ILs. Lignin is the second most abundant naturally occurring polymer next to cellulose, which represents a significant component of carbon on earth. Large amount of technical lignins such as Kraft lignin and lignosulfonate is produced as by-products in the pulp and paper industries. It is also expected that more lignin will become available in coming years as the production capability of second generation of biofuels increases. As a renewable and resource, lignin and lignin derived products (phenolic) are an important material. DESs with lignin-derived phenolic compounds either as a single monomer or phenolic mixture can be used in the present invention.

The one-pot biomass pretreatment, saccharification, and fermentation with bio-compatible deep eutectic solvents (DESs). The used bio-compatible DESs are tested for microbial, such as yeast, compatibility and toxicity. The pretreatment efficacy of the selected DESs are tested. The uses of the DESs for biomass processing eliminates the need to remove any solvent after biomass pretreatment, thus making the one-pot approach possible.

In some embodiments, the biomass is a lignocellulosic biomass. In some embodiments, the vessel is made of a material that is inert, such as stainless steel or glass, which does not react or interfere with the reactions in the one-pot composition.

In some embodiments, the pretreatment comprises about 0.5 g of biomass (such as corn stover) mixed thoroughly with about 4.5 g DES (such as choline chloride and glycerol) in a suitable inert vessel, such as a glass vessel, followed by heating up to about 180° C. and the temperature is maintained for a suitable period of time, such as about 2 hours. After pretreatment, the resulting slurry is cooled to below about 50° C. and is immediately ready for the following saccharification and microbial conversion. The saccharification is carried out with a suitable enzyme, such as a commercial enzyme mixture (for example, CTec2 and HTec2 from Novozymes A/S (Bagsværd, Denmark), at about 50° C. and about pH 5 at about 48 rpm in an incubator with shaking function. After a suitable period of time, such as about 48 hours, of saccharification, the generated sugar stream is then immediately ready for microbial conversion. For example, a wild-type yeast, such as Saccharomyces cerevisiae, is inoculated at temperature (about 30° C. to about 37° C.) for anaerobic ethanol fermentation.

In some embodiments, using bio-compatible DESs enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

In some embodiments, ethanolaminium acetate [Eth][OAc] is a PIL that is recoverable/recyclable via distillation.

In some embodiments, the acid—base conjugate ILs (such as,.1,1,3,3-tetramethylguanidinium propionate [TMGH][CO2Et])64 and 1,5-diazabicyclo[4.3.0]non-5-enium propionate ([DBNH][CO2Et])102) are synthesized for efficient biomass treatment. Their distillable characteristic is attributed to the higher acidity of the cations, allowing for the dissociation of ILs to the neutral acid and base IL precursors at elevated temperatures. For example, when being heated to a temperature about 100-200° C. under reduced pressure (1 mmHg), the [TMGH][CO2Et] is dissociated into the 1,1,3,3-tetramethylguanidine (TMG) and carboxylate (HCO2Et).

For the distillable ILs-protic ILs an equilibrium exists between the IL precursors and IL:

B:₍ₗ₎+A−H₍ₗ₎ [symbol] _{[B-H}⁺][A⁻]₍ₗ₎

B:₍ₗ₎ [symbol] B:_{(g)}, A−H₍ₗ₎ [symbol] A−H_{(g)}

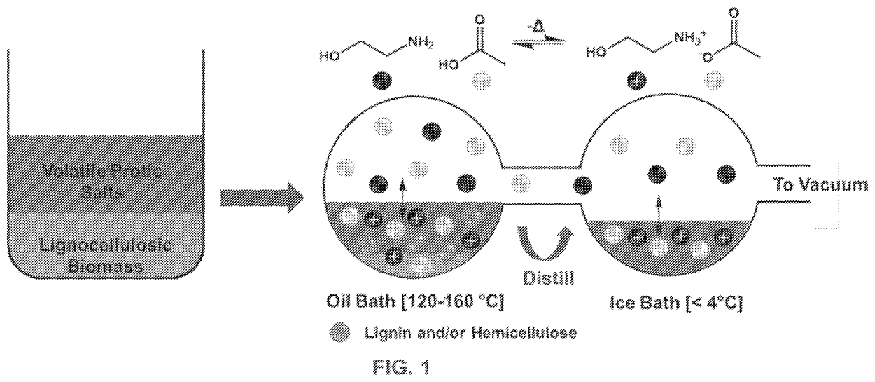

The equilibrium can be pushed to either side based on reaction coordinates, such as pressure, temperature, or the like. FIG. 1 shows an embodiment for the recovery of distillable IL/solvent.

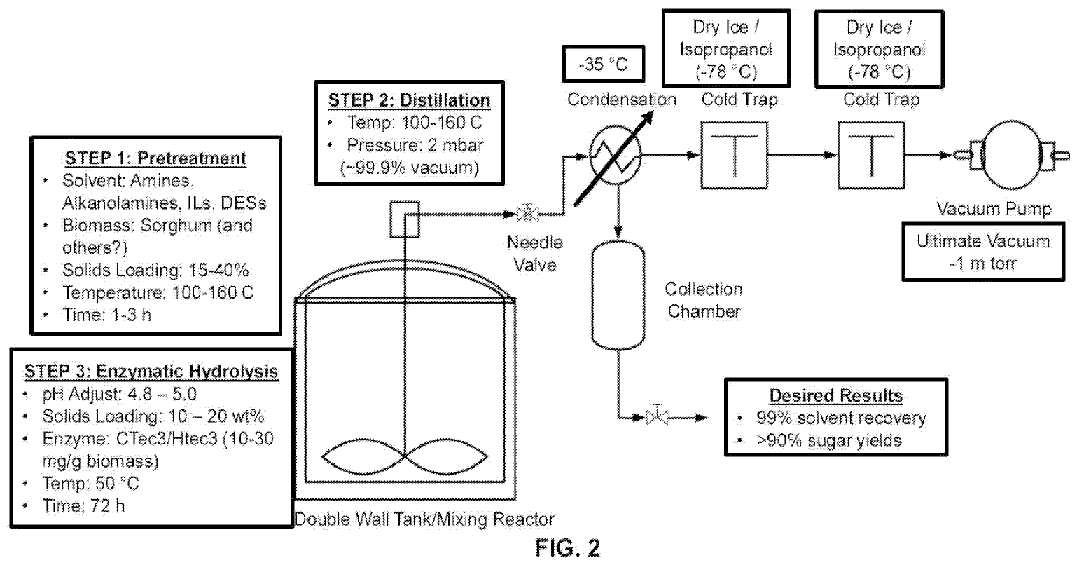

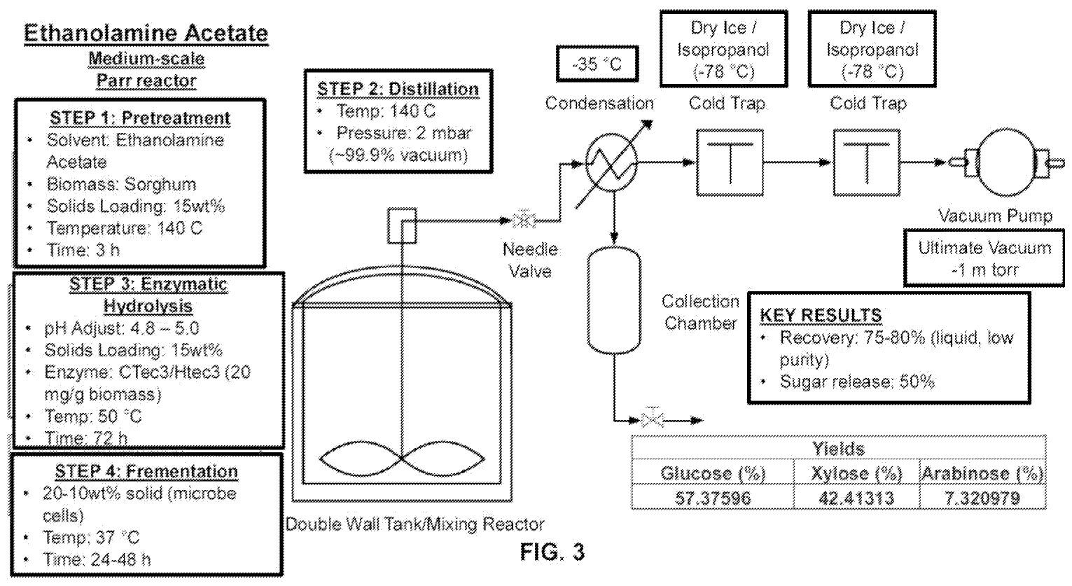

FIG. 2 shows an embodiment of a method/system for the recovery of distillable IL/solvent used in a biomass pretreatment system. FIG. 3 shows another embodiment of a method/system for the recovery of distillable IL/solvent used in a biomass pretreatment system.

### Deep Eutectic Solvent (DES)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 16 ° C., or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing step(s) produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps described herein are continuous, the one-pot composition is in a steady state.

In some embodiments, the introducing step comprises heating the mixture comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtherias, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandá; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populusxcanadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C4 or C3 grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

### EXAMPLE 1

### Distillable Ionic Liquids/Deep Eutectic Solvents for an Effective Recycling and Recovery Approach

Pretreatment using ionic liquids (ILs) is one of the most effective methods for producing high yields of lignin and biomass-derived fermentable sugars, however, there are few economical methods for IL recovery/recycling. This reality has hitherto limited the commercial potential of this process; therefore, it is crucial to develop recyclable ILs as green solvents for biomass pretreatment. Our current research focuses on developing recyclable IL pretreatment technologies while simultaneously facilitating the efficient depolymerization of both polysaccharides and lignin. This process is primarily enabled by utilizing a recycling method (based on distillation) that can be readily integrated with the pretreatment process for a one-pot/consolidated approach.

Developing a low-cost and high efficiency lignocellulosic biomass deconstruction process is a critical step towards the widespread adoption of lignocellulosic biofuels. Ionic liquids (ILs) and deep eutectic solvents (DESs) are novel alternative solvents for biomass pretreatment and conversion, and they are most notably one of the most effective methods for producing lignin and high yields of fermentable sugars for bioenergy production. Despite their commercial potential, the cost of IL/DES utilization (typically associated with their synthesis, purification and reuse/recycling) is a significant problem that must be addressed before an affordable IL/DES-based process is commercially viable. Therefore, this study features the use of distillable solvents for the development of an integrated biomass pretreatment approach that combines effective pretreatment with a simplistic and energy efficient recovery/recycling method.

Protic ionic liquids (PILs) that are formed with the combination of organic ammonium-based cations and organic carboxylic acid-based anions are an attractive group of solvents worth considering for this process. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application.

The PIL—hydroxyethylammonium acetate—[Eth][OAc]—has already been demonstrated as an effective solvent for biomass pretreatment and is also relatively cheap due to its ease of synthesis.¹ Therefore, this PIL and chemically analogous PILs were studied for their distillability, as well as, their effect on biomass deconstruction in a one-pot/consolidated process. Preliminary results indicate a PIL recovery of 98% for “neat” IL distillation of [Eth][OAc]), followed by PIL recovery [˜80-85%] after biomass pretreatment with 15% biomass loading. Following the PIL removal, the residual biomass was saccharified to generate ˜74% total sugars (compared to ˜78% sugars for ˜One pot and ˜91% sugars—Early separation).

This is a promising proof of concept that supports our approach for distilling ionic liquids as a recovery method. Once optimized, this will launch our research into economic regimes, making an IL-based biorefinery a realizable goal.

Sun, J.; Konda, N. V. S. N. M.; Parthasarathi, R.; Dutta, T.; Valiev, M.; Xu, F.; Simmons, B. A.; Singh, S. One-Pot Integrated Biofuel Production Using Low-Cost Biocompatible Protic Ionic Liquids. Green Chem. 2017, 19 (13), 3152-3163.

### EXAMPLE 2

### Distillable Ionic Liquids/Deep Eutectic Solvents for an Effective Recycling and Recovery Approach

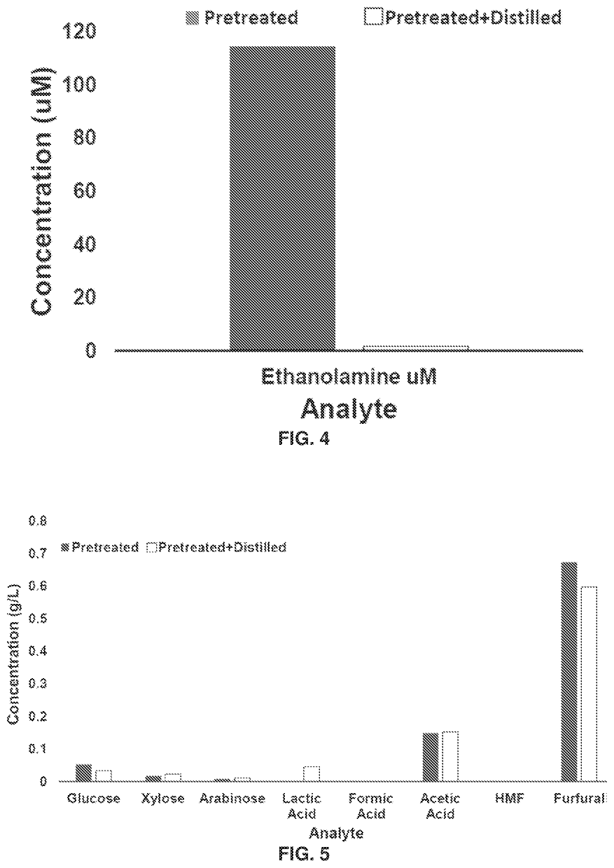

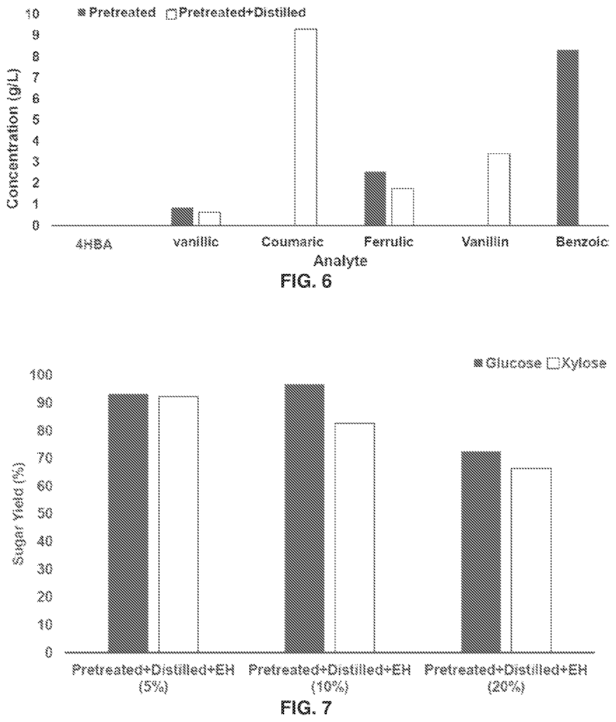

Distillation efficiency is measured using the following procedure: pretreat at 100° C. for 1 hour; distill at 100° C. for 1 hours in full vacuum, 40% solid loading (SL) with ethanolamine, enzyme saccharification (5%, 10%, 20% SL), and 10 mg/g biomass of CTEC/HTEC 9/1 cellulase. Distillation efficiency can be measured by determining the concentration of pretreating solvent in the solids after pretreatment. FIG. 4 shows the removal of the solvent by distillation is about 98.7%. FIG. 5 shows the analysis of sugars and acids in solids after distillation. FIG. 6 shows the analysis of aromatics in solids after distillation.

FIG. 7 shows the sugar release after pretreatment, distillation and saccharification in a variety of percentages of solid loading. The procedure comprises: Opretreat at 100° C. for 1 hour; distill at 100° C. for 1 hours in full vacuum, 40% SL with ethanolamine, enzyme saccharification (5%, 10%, 20% SL), and 10 mg/g biomass of CTEC/HTEC 9/1 cellulase.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to produce a sugar from a biomass, the method comprising:
(a) providing a first mixture comprising a solubilized biomass and a distillable acid-base conjugate salt (DABCS) or deep eutectic solvent (DES), wherein (i) the DABCS is a protic ionic liquid (PIL) or a protic salt comprising a DABCS cation and a DABCS anion, and (ii) the DES is any combination of Lewis or Brønsted acid and base comprising any anionic and/or cationic species that have sufficient vapor pressure so that it can be readily distilled;
(b) distilling at least part of the DABCS from the first mixture in order to separate the at least part of the DABCS from the first mixture;
(c) optionally introducing an enzyme and/or a microbe to the first mixture such that the enzyme and/or microbe produce a sugar from the solubilized biomass; and,
(d) optionally the sugar is separated from the first mixture.

2. The method claim 1, further comprising (e) introducing at least part of the DABCS separated in the (b) distilling step to the first mixture in step (a).

3. The method claim 1, further comprising (f) introducing more biomass to the first mixture in step (a).

4. A first mixture comprising a biomass and a distillable acid-base conjugate salt (DABCS) comprising a DABCS cation, and a DABCS anion.

5. A system comprising a reactor vessel, a condenser, a collection chamber, one or more cold traps, and low pressure source; wherein the reactor vessel is in fluid communication with a condenser, and the condenser is in fluid communication with (i) the collection chamber and (ii) the low pressure source via the one or more cold traps; and wherein the reactor vessel contains the first mixture of claim 4.
